(19) United States
(12) Patent Application Publication
(10) Pub. No.: US 2024/0024692 A1
(43) Pub. Date: Jan. 25, 2024

(54) SYSTEM AND METHOD FOR A WEARABLE PAIN TREATMENT DEVICE

(71) Applicants: James Shiraz KHAN, Toronto (CA); Vivek SAINI, Mississauga (CA)

(72) Inventors: James Shiraz KHAN, Toronto (CA); Vivek SAINI, Mississauga (CA)

(21) Appl. No.: 18/355,301

(22) Filed: Jul. 19, 2023

Related U.S. Application Data
(60) Provisional application No. 63/390,308, filed on Jul. 19, 2022.

(51) Int. Cl. A61N 2/00; A61N 2/02

(52) U.S. Cl. CPC A61N 2/006; A61N 2/008; A61N 2/02

(57) ABSTRACT

A peripheral nerve stimulation through magnetic fields using a wearable, non-invasive, and mobile device using targeted magnetic stimulation. The magnetic stimulation system can be used to target different areas of pain including back pain, breast pain and knee pain. The device will be battery powered and include electronics to generate a varying current through a coil which will emit a varying magnetic field. This varying magnetic field freely travels through human tissue and couples with peripheral nerves which are electrically conductive.

### CROSS REFERENCE TO RELATED APPLICATIONS

The application claims priority to and the benefit of U.S. patent application Ser. No. 63/223,997 entitled “SYSTEM AND METHOD FOR A WEARABLE PAIN TREATMENT DEVICE” filed on Jul. 19, 2022, the disclosure of which is incorporated herein by reference in its entirety.

### BACKGROUND

The embodiments described herein relate to pain treatment, in particular technologies related to pain treatment using a wearable device.

According to studies, more people in the world live with pain or chronic pain than cancer, heart disease, and diabetes combined. Pain is strongly associated with decreased function, poor quality of life, sleep impairment, substance abuse, depression, and increased suicide risk. This results in over $600B in direct (healthcare expenditures) and indirect (loss of productivity) costs in the US alone. Unfortunately, there is no cure for pain and current treatment options are ineffective or associated with side-effects. Significant knowledge gaps remain in both the causes and treatments of pain conditions.

Peripheral nerves carry pain signals from the periphery and are involved in a large proportion of pain disorders. Given that nerves are electrically conductive, it is possible to use external electromagnetic fields to either stimulate or inhibit neuronal function. This technique is emerging as a novel and innovative approach to treat several disorders of the nervous system (i.e., Transcranial Magnetic Stimulation [TMS] in the management of depression).

Non-invasive magnetic stimulation may result in reduction in pain after a single session. Prior literature suggest that peripheral nerve magnetic stimulation can provide analgesia through activation of endogenous inhibitory mechanisms, recruitment of peripheral non-nociceptive afferent fibers (closing the pain gate), and modulation of the autonomic nervous system and central cortical/subcortical areas.

There is a desire to use non-invasive magnetic stimulation solutions to treat chronic pain.

### SUMMARY

A wearable, non-invasive and mobile device and system using targeted magnetic stimulation for peripheral nerve stimulation through magnetic fields. The magnetic stimulation system can be used to target different areas of pain including back pain, breast pain and knee pain. The device will be battery powered and include electronics to generate a varying current through a coil which will emit a varying magnetic field. This varying magnetic field freely travels through human tissue and couples with peripheral nerves which are electrically conductive.

### BRIEF DESCRIPTION OF THE DRAWINGS

FIG. 1 is a block diagram illustrating components of a system for a wearable and non-invasive magnetic stimulation system.

FIGS. 2A to 2C are diagrams illustrating exemplary waveforms.

FIG. 3 is a diagram illustrating different form factors of the magnetic stimulation system.

FIGS. 4A and 4B are diagrams illustrating a magnetic stimulation system targeting back pain.

FIGS. 5A and 5B are diagrams illustrating a magnetic stimulation system targeting breast pain.

FIGS. 6A and 6B are diagrams illustrating a magnetic stimulation system targeting knee pain.

FIGS. 7A and 7B are diagrams illustrating how a magnetic stimulation system works.

FIG. 8 is a chart illustrating experimental data.

### DETAILED DESCRIPTION

According to this disclosure, a mobile, non-invasive, wireless, wearable, neuromodulation system can provide effective pain management through focused magnetic fields resulting in peripheral nerve stimulation (mPNS). The device will be battery-powered and include electronics to generate a varying current through a coil which will emit a varying magnetic field. This varying magnetic field freely travels through human tissue and couples with peripheral nerves which are electrically conductive.

### Magnetic Stimulation System

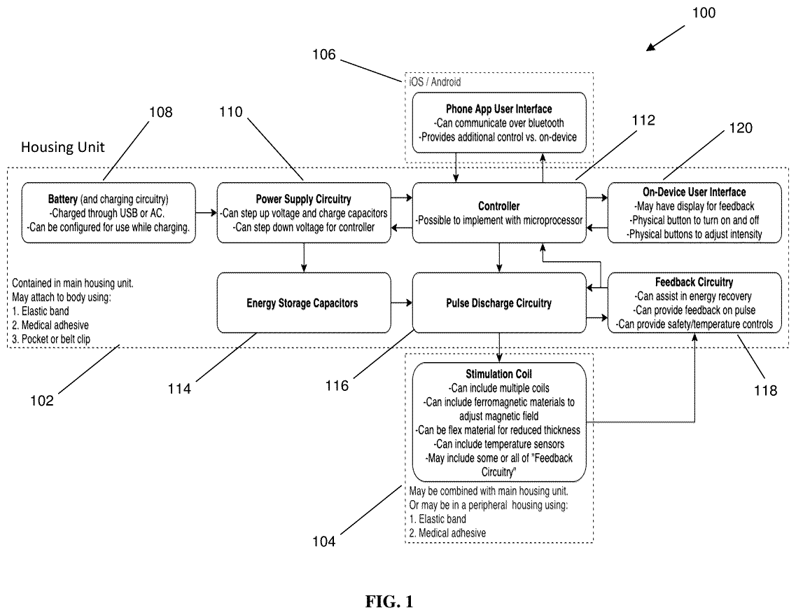

FIG. 1 is a block diagram illustrating components of an exemplary embodiment of a system for a wearable and non-invasive magnetic stimulation system. According to FIG. 1, the magnetic stimulation system 100 consists of a main housing unit 102, one or more stimulation coils 104 and an application to manage controls, located on either the housing unit 102 and/or on a separate mobile IOS or Android application 106.

According to FIG. 1, housing unit 102 consists of a battery 108, a power supply circuitry 110, a controller 112 or microprocessor, energy storage capacitors 114, a pulse discharge circuitry 116, feedback circuitry 118 and a device user interface 120. The battery 108 can be charged via USB or AC current and can be configured to be used while charging. The power supply circuitry 110 can step up the voltage and charge the energy storage capacitors 114 and step down the voltage for connection to the controller 112.

The feedback circuitry 118 assists in energy recovery and provides feedback on a pulse and provides safety and/or temperature controls. The device user interface 120 may have one or more light emitting diode (LED) or liquid crystal display (LCD) to provide the user with visual feedback, physical buttons to turn on/off the device and a button to adjust and control the stimulation.

The controller 112 or microprocessor of the main housing unit 108 also contains communication components (not shown) that enables the main housing unit to communicate wireless via cellular connectivity, Wi-Fi® connectivity or Bluetooth® connectivity with a mobile device or the internet.

The magnetic stimulation system 100 of FIG. 1 also consists of stimulation coils 104 that receive inputs from the pulse discharge circuitry and provide output to the feedback circuitry. The stimulation coil may include a single or multiple coils and may include ferromagnetic materials to adjust the strength of the magnetic field. The stimulation coil may also include one or more temperature sensors and may be constructed of flex material to reduce thickness. According to some embodiments, the stimulation coil may be combined with the main housing unit (vs a separate peripheral housing unit) which can be attached by elastic band or medical adhesive to the user. The stimulation coil and main housing unit may be incorporated into a wearable apparatus, such as a knee brace for knee stimulation or a bra for breast stimulation. The stimulation coil may be incorporated into a handheld unit, either on its own or alongside the main housing unit.

According to FIG. 1, in one embodiment, the magnetic stimulation system 100 may also include an iOS or Android or other mobile application 106 on a mobile phone or computing tablet. The mobile application 106 has a graphical user interface that can communicate over Bluetooth with the main housing unit and provides additional control on the mobile devices in addition to the on-device controls. According to the disclosure, FIG. 1 is one exemplary embodiment of the disclosure; other implementations may also be contemplated by a person skilled in the art.

In some situations, a signal with a sufficiently short pulse rise time (<=100 um) and sufficiently high magnetic field strength (>=0.1 T) may cause neuromodulation and subsequently pain reduction. The device can generate different waveforms, but the current focus is on a monophasic pulse with quick bursts (>=10 Hz) separated into multiple cycles (<=10 Hz). The quick bursts with short rise time provide a high dB/dT (change in magnetic field) to elicit a neuropathic response while the shorter frequency cycles allow for smaller electronics and thermal cooling. Ine one embodiment, the battery life of the device is sufficient for at least a 10-minute treatment. In further embodiments, the power source can be connected to an AC adapter to provide a continuous power source whereby the treatment can be extended to 60 minutes, 120 minutes or even 240 minutes.

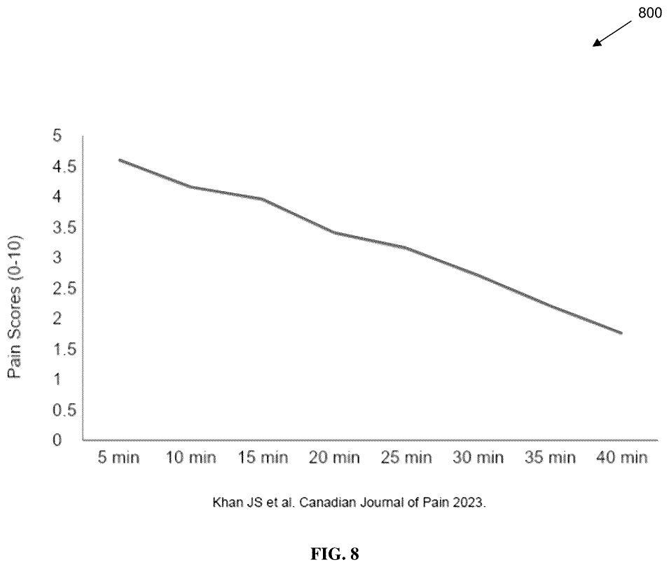

The main housing unit 108 utilizes a FIG. 8 coil for a deeper focused field for certain pain condition (e.g., mononeuropathies, nerve root compressions) or a single circular coil for a wider magnetic field for other pain conditions (e.g., facet arthropathy/non-specific back pain). The coil may be made of a copper coil for lower cost and lower resistive losses, however a flex coil design which allows for a thinner form factor may also be used.

The main housing unit 108 may be attached to the body of a user using medical grade adhesive, sticky silicone, elastic strap, a pocket, belt clip or built into clothing or other suitable connection mechanism known to a person skilled in the art. The electronics and coil may be in one unit. For example, the electronics may be behind the coil with optional shielding for EMI protection, or the electronics may be adjacent to the coil. The electronics and coil may also be in two separate units connected by wire or flex. In one embodiment, the main housing may be insulated with foam or other sound dampening materials to provide for a quieter treatment.

### Waveforms

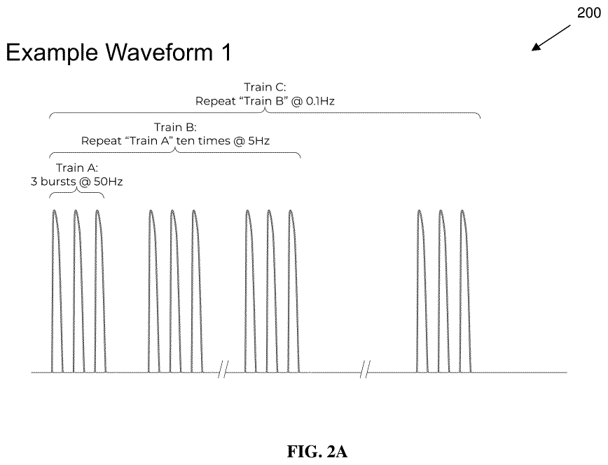

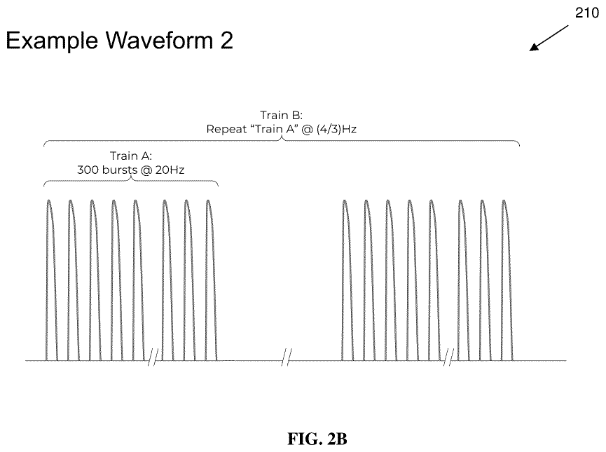

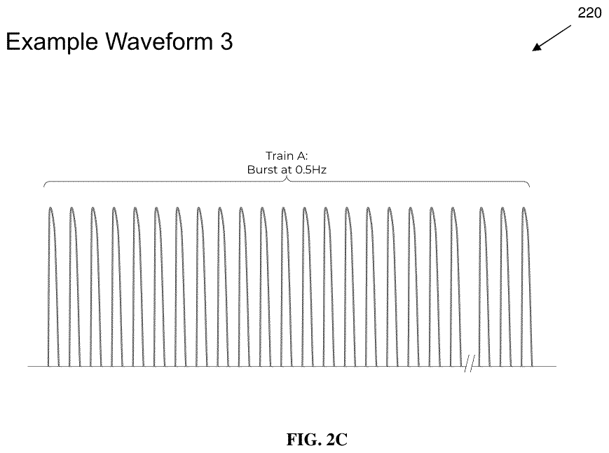

FIGS. 2A to 2C are diagrams illustrating exemplary waveforms. According to FIG. 2A, diagram 200 illustrates waveforms (i.e., Example Waveform 1) performing three frequencies at once. One burst is first repeated several times at a high frequency (3× @ 50 Hz pictured below), and then that “pulse train” is repeated at a slower frequency (10× @ 5 Hz pictured below), and that whole sequence repeats at an even slower frequency (10× @ 0.1 Hz pictured below).

In one embodiment, other waveforms (i.e., Example Waveform 2) such as a 300× @ 20 Hz, repeated at 4/3 Hz waveform (i.e., 20 Hz for 15 seconds followed by a 30 second break) shown as diagram 210 of FIG. 2B and/or a 0.5 Hz constant waveform (i.e., Example Waveform 3) shown as diagram 220 in FIG. 2C, may also be implemented.

According to the disclosure, the exact effects of frequency on the body are not well understood but clinically 1-100 Hz may be effective with different people or regions of the body responding better to different frequencies. A person skilled in the art may also implement the proposed disclosure at higher frequencies, that is, greater than 100 Hz. Running two frequencies as shown allows the system to capture potential benefits of either. The slowest frequency mainly functions as an electronic buffer and to keep the device thermally cool.

According to this disclosure, the waveform is also specifically optimized to elicit an electrical current in the nerves (similar to a TMS device). To elicit an electrical current in the nerves, a pulse rise time <100 us (for a high dB/dt) and a magnet strength >0.1 mT (sufficient amplitude) may be required. The system is optimized around having larger electronics (capacitors primarily) vs. battery to allow for the high pulse rise time and magnetic strength vs. competitors who optimize around long battery life.

The magnetic stimulation system of this disclosure is a combination wearable and mobile peripheral nerve stimulator that provides sufficient pulse rise time and magnetic strength as a magnetic peripheral stimulator.

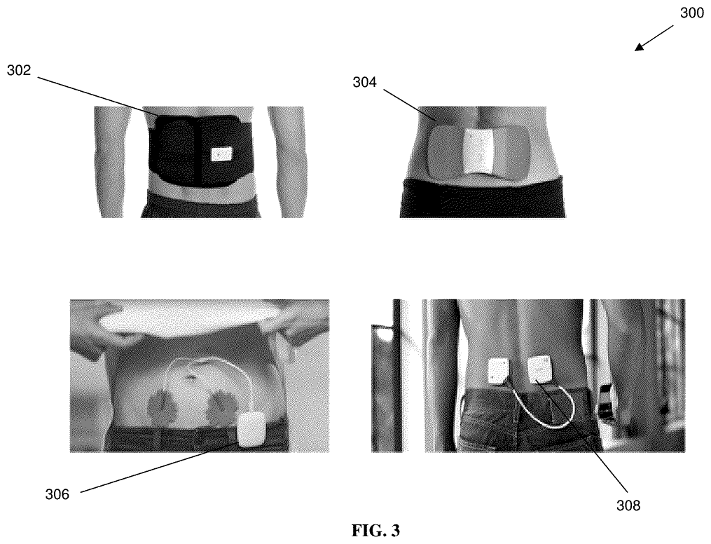

FIG. 3 is a diagram illustrating different form factors of the magnetic stimulation system. According to FIG. 3, the system can be housed in different form factors. The first form factor 302 includes an elastic band for the device. The second form factor 304 is a medical adhesive for device on a single unit 304. The third form factor 306 includes a medical adhesive for coil with electronic unit and battery in separate unit (i.e., in pocket or belt clip). Lastly a fourth for factor 308 includes a medical adhesive for coil with medical adhesive for electronic unit and battery on separate unit.

According to some embodiments of this disclosure, the coils can be intelligently placed in a strategic location such that the magnetic stimulation happens along the nerve pathways associated with the specific pain whereby neuromodulation, targeting specific pain targets, can be performed.

### Back Pain

Most back pain in people from 30 to 50 years of age will be myofascial pain from the lumbar paraspinal muscles. Most back pain in people 50+will be due to pain from facet arthropathy (arthritis of the facet joints). Both the facet joints and paraspinal muscles are innervated by the lumbar medial branch nerves, located at the junction between the superior articular process and transversus process at each lumbar level.

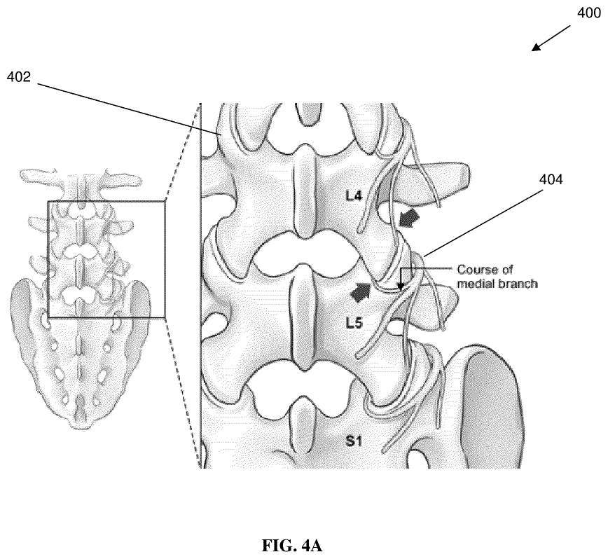

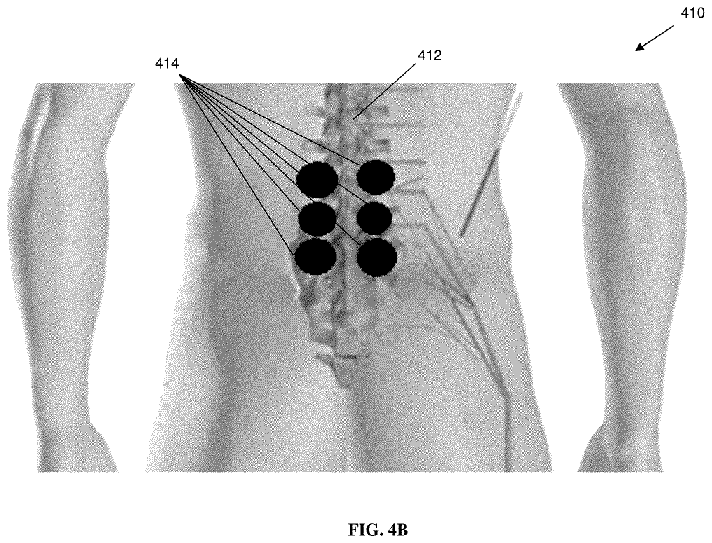

FIGS. 4A and 4B are diagrams illustrating a magnetic stimulation system targeting back pain. According to FIG. 4A shows diagram 400 illustrating magnified view of L4 and L5 of spine 402 and medial branch nerve 404.

According to FIG. 4B, diagram 410 illustrates a plurality of magnetic stimulators 414 placed on lumbar spine 412. Repetitive pulsed peripheral magnetic stimulation is provided to the midline of the lumbar spine 414 to stimulate the medial branch nerves.

### Breast Pain

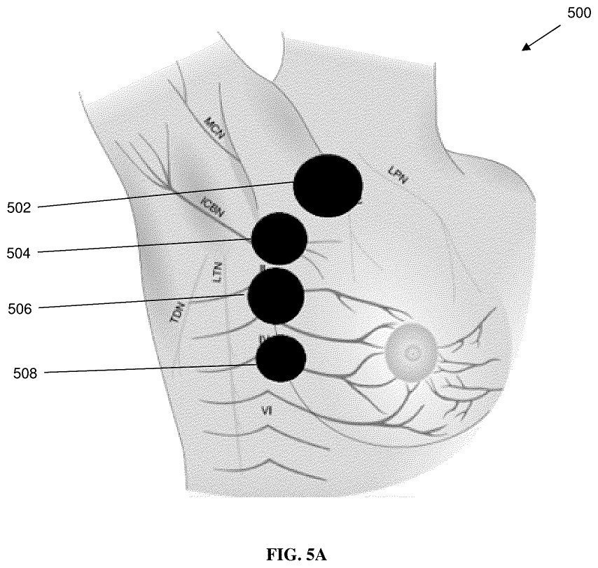

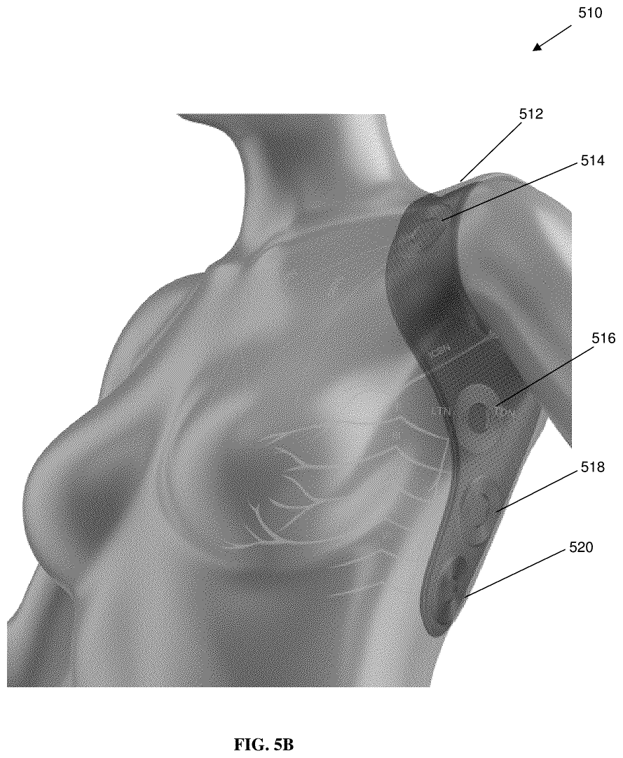

The innervation of the breast is quite complex and involves involvement of the medial and lateral pectoral nerves, axillary innervation with the intercostobrachial nerve, and intercostal nerves. FIGS. 5A and 5B are diagrams illustrating a magnetic stimulation system targeting breast pain.

According to FIG. 5A, diagram 500 illustrates placement of magnetic stimulators 502, 504, 506 and 508 placed adjacent to the medial pectoral nerve, the lateral pectoral nerve, the intercostobrachial nerve and the intercostal nerve respectively.

FIG. 5B is a diagram illustrating an exemplary magnetic stimulation apparatus targeting breast pain. According to FIG. 5B, diagram 510 illustrates a shoulder mounted magnetic stimulation apparatus 512 having a plurality of magnetic stimulators 514, 516, 518 and 520. The magnetic stimulators provide magnetic stimulation to the medial pectoral nerve, the lateral pectoral nerve, the intercostobrachial nerve and the intercostal nerve respectively.

### Knee Pain

The innervation of the knee joint includes the anterior knee joint innervated, in all specimens, by articular branches from Nerve to Vastus Intermedius (NVI), Never to Vastus lateralis (NVL), Nerve to Vastus Medialis (NVM), Superior lateral genicular nerve (SLGN), Inferior lateral genicular nerve (ILGN), Superior medial genicular nerve (SMGN), Inferior medial genicular nerve (IMGN), common fibular nerve (CFN), and recurrent fibular nerve (RFN).

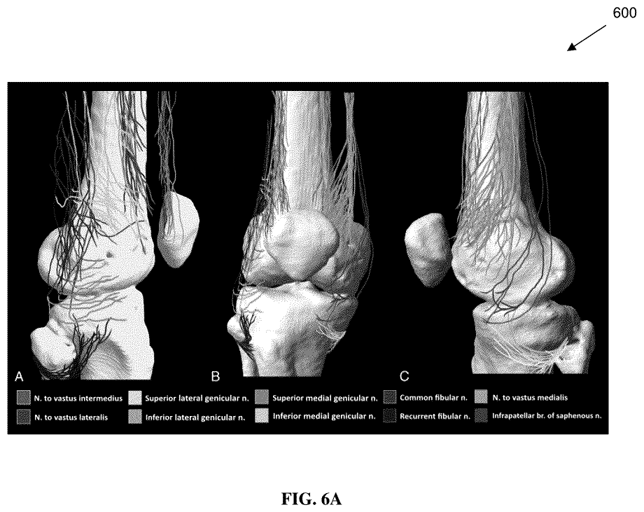

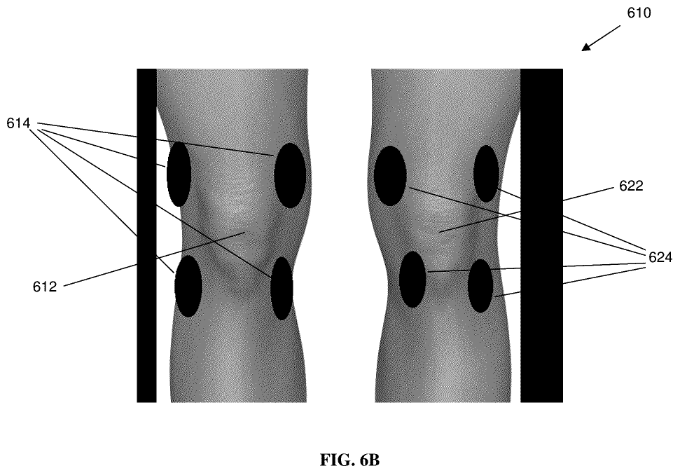

FIGS. 6A and 6B are diagrams illustrating a magnetic stimulation system targeting knee pain. According to FIG. 6A, diagram 600 is shown illustrating images of the nerves innervating the knee. According to FIG. 6B, diagram 610 illustrates magnetic stimulators 614 and 622 placed around the left knee 612 and right knee 622.

### How it Works

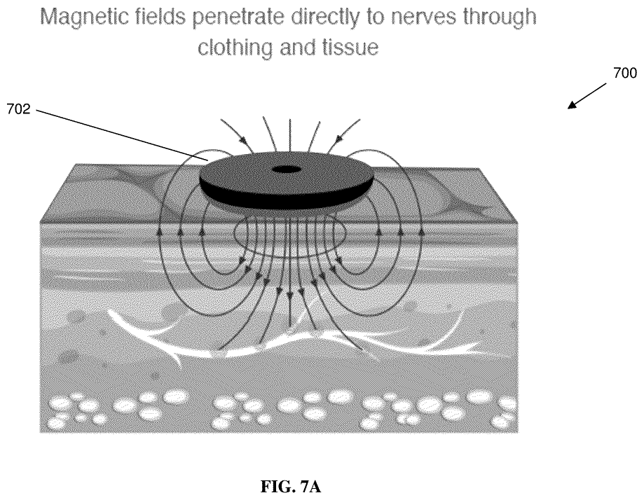

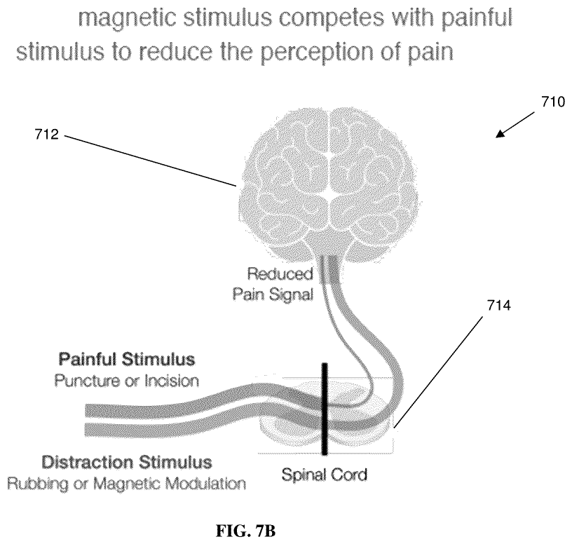

FIGS. 7A and 7B are diagrams illustrating how a magnetic stimulation system works. According to FIG. 7A, diagram 700 comprises a magnetic stimulator 702 placed on the skin of the patient. Magnet stimulator 702 produces magnetic fields that penetrate directly to nerves through clothing and tissue of the patient.

Magnetic stimulus competes with painful stimulus to reduce the perception of pain. FIG. 7B is a diagram illustrating how the brain perceives pain. According to FIG. 7B, diagram 710 shows brain 712 and spinal cord 714. According to FIG. 7B, painful stimulus (e.g., puncture or incision) is sent as signals through the spinal cord 714 nerves to the brain 712. Distraction stimulus (e.g., rubbing, scratching or magnetic stimulus modulation) are also sent from the spinal cord 714 nerves to the brain 712 to provide a distraction and provide a perception of reduced pain signal.

FIG. 8 is a chart illustrating experimental data. According to FIG. 8, chart 800 illustrates the reduction of pain (pain scores) over time (in minutes) as magnetic stimulation is applied. This is supported by research by 'Park et al., “PMS for chronic pain after surgery: systematic review and meta-analysis”, Journal of Pain. 2023″ which is incorporated by reference in its entirety. The Park et al reference also states that acute post-operative pain is reduced after 1-2 months post-surgery with magnetic stimulation treatment.

According to the disclosure, a wearable magnetic stimulation apparatus for peripheral nerve stimulation using magnetic fields for non-invasive pain treatment is disclosed. The apparatus comprises one or more stimulation coils and a housing unit. The housing unit further comprises a power supply, power supply circuitry, a controller or microprocessor, an energy storage capacitor, a pulse discharge circuitry, a feedback circuitry and a device user interface.

According to the disclosure, one or more stimulation coils are configured to generate a varying current through the coils which will emit varying magnetic fields to stimulate the peripheral nerves. The power supply is a battery or an AC adapter. The battery can be charged via USB or AC current, and the apparatus can be configured to be used while charging.

According to the disclosure, the apparatus further comprises an application to manage controls; and a mobile application stored in memory of the housing unit or on a separate application on a mobile device. The mobile application has a graphical user interface (GUI) that can communicate over Bluetooth® with the housing unit and provides additional controls on a mobile device.

According to the disclosure, the power supply circuitry of the apparatus can step up the voltage and charge the energy storage capacitors and step down the voltage for connection to the controller. The feedback circuitry of the apparatus assists in energy recovery and provides feedback on a pulse and provides safety or temperature controls.

According to the disclosure, the device user interface of the apparatus further comprises light emitting diodes (LED) or a liquid crystal display (LCD) to provide the user with visual feedback, physical buttons to operate device and buttons to adjust and control the stimulation. The apparatus further comprises communication components that enable the housing unit to communicate wirelessly via cellular connectivity, WiFI® connectivity or Bluetooth® connectivity with a mobile device or the internet.

According to the disclosure, the stimulation coils of the apparatus receive input from the pulse discharge circuitry and provide output to the feedback circuitry. The stimulation coils further comprise a single or multiple coils and include ferromagnetic materials to adjust the strength of the magnetic field. The housing unit of the apparatus further comprises a temperature sensor, the temperature sensor constructed of a flexible material to reduce thickness.

According to the disclosure, a method of using a magnetic stimulation apparatus for peripheral nerve stimulation for pain treatment is disclosed. The method further comprises the step of placing a magnetic coil of the apparatus in an area for pain treatment, powering on the apparatus, generating magnetic pulses and providing the magnetic pulses to the treatment area for a treatment duration. The magnetic waveforms from the apparatus are configured to illicit a response from the body.

According to the disclosure, the method further comprises the step of selecting different modes for treatment duration, wherein the different modes are user configurable. The treatment duration is selected between a range of 20 minutes to 120 minutes.

According to the disclosure, the response of the method can inhibit pain, tingling or sensory stimulation from the user. The method further comprises the step of powering off the apparatus upon completion of the treatment or upon expiration of the duration. The method further comprises the step of wearing the apparatus in the area for treatment.

According to the disclosure, a wearable magnetic stimulation apparatus for treatment of breast pain using magnetic fields for peripheral nerve stimulation is disclosed. The apparatus comprises a housing unit. The housing unit further comprises a power supply, power supply circuitry, a controller or microprocessor, an energy storage capacitor, a pulse discharge circuitry, a feedback circuitry and a device user interface. The apparatus further comprises a holster configured to fit around shoulder and chest, a cable connecting the housing unit to holster and one or more stimulation coils in the holster.

The stimulation coils are configured to stimulate the lateral or medial pectoral nerves, the intercostal brachial nerve (ICBN), the long thoracic nerve (LTN) or the thoracodorsal nerve (TDN) and the intercostal nerves. The stimulation coils are placed in a location that is in proximity to the skin to provide optimal stimulation. Furthermore, the one or more stimulation coils are configured to generate a varying current through the coils which will emit varying magnetic fields to stimulate the peripheral nerves. The holster of the apparatus is padded with adhesive.

According to the disclosure, the magnetic stimulation apparatus can be used to target different areas of pain including back pain, breast pain and knee pain. Furthermore, a plurality of pulse sequences of the apparatus is generated that can be configured for the type of treatment.

The functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor. A “module” can be considered as a processor executing computer-readable code.

A processor as described herein can be a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, or microcontroller, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, any of the signal processing algorithms described herein may be implemented in analog circuitry. In some embodiments, a processor can be a graphics processing unit (GPU). The parallel processing capabilities of GPUs can reduce the amount of time for training and using neural networks (and other machine learning models) compared to central processing units (CPUs). In some embodiments, a processor can be an ASIC including dedicated machine learning circuitry custom-build for one or both of model training and model inference.

The disclosed or illustrated tasks can be distributed across multiple processors or computing devices of a computer system, including computing devices that are geographically distributed. The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.” While the foregoing written description of the system enables one of ordinary skill to make and use what is considered presently to be the best mode thereof, those of ordinary skill will understand and appreciate the existence of variations, combinations, and equivalents of the specific embodiment, method, and examples herein. The system should therefore not be limited by the above-described embodiment, method, and examples, but by all embodiments and methods within the scope and spirit of the system. Thus, the present disclosure is not intended to be limited to the implementations shown herein but is to be accorded the widest scope consistent with the principles and novel features disclosed herein.

What is claimed:

1. A wearable magnetic stimulation apparatus for peripheral nerve stimulation using magnetic fields for non-invasive pain treatment, the apparatus comprising:
one or more stimulation coils;
a housing unit, the housing unit further comprising:
a power supply;
power supply circuitry;
a controller or microprocessor;
an energy storage capacitor;
a pulse discharge circuitry;
a feedback circuitry; and
a device user interface;
wherein the one or more stimulation coils are configured to generate a varying current through the coils which will emit varying magnetic fields to stimulate the peripheral nerves.

2. The apparatus of claim 1 wherein the power supply is a battery or an AC adapter.

3. The apparatus of claim 2 wherein the battery can be charged via USB or AC current, and the apparatus can be configured to be used while charging.

4. The apparatus of claim 1 further comprising:
an application to manage controls; and
a mobile application stored in memory of the housing unit or on a separate application on a mobile device.

5. The apparatus of claim 6 wherein the mobile application has a graphical user interface (GUI) that can communicate over Bluetooth® with the housing unit and provides additional controls on the mobile device.

6. The apparatus of claim 1 wherein the power supply circuitry can step up the voltage and charge the energy storage capacitors and step down the voltage for connection to the controller.

7. The apparatus of claim 6 wherein the feedback circuitry assists in energy recovery and provides feedback on a pulse and provides safety or temperature controls.

8. The apparatus of claim 1 wherein the device user interface further comprises light emitting diodes (LED) or a liquid crystal display (LCD) to provide the user with visual feedback, physical buttons to operate device and buttons to adjust and control the stimulation.

9. The apparatus of claim 1 further comprises communication components that enable the housing unit to communicate wireless via cellular connectivity, WiFI® connectivity or Bluetooth® connectivity with a mobile device or the internet.

1. aratus of claim 1 wherein the stimulation coils receive input from the pulse discharge circuitry and provide output to the feedback circuitry.

11. The apparatus of claim 1 wherein the stimulation coils further comprise a single or multiple coils and include ferromagnetic materials to adjust the strength of the magnetic field.

12. The apparatus of claim 1 wherein the housing unit further comprises a temperature sensor, the temperature sensor constructed of a flexible material to reduce thickness.

13. A method of using a magnetic stimulation apparatus for peripheral nerve stimulation for pain treatment, comprising the step of:
placing a magnetic coil of the apparatus in an area for pain treatment;
powering on the apparatus;
generating magnetic pulses; and
providing the magnetic pulses to the treatment area for a treatment duration;
wherein magnetic waveforms from the apparatus are configured to illicit a response from the body.

14. The method of claim 13 further comprising the step of selecting different modes for treatment duration, wherein the different modes are user configurable.

14. The method of claim 14 wherein the treatment duration is selected between a range of 20 minutes to 120 minutes.

16. The method of claim 13 wherein the response can inhibit pain, tingling or sensory stimulation from the user.

17. The method of claim 13 further comprising the step of powering off the apparatus upon completion of the treatment or upon expiration of the duration.

18. The method of claim 13 further comprising the step of wearing the apparatus in area for treatment.

19. A wearable magnetic stimulation apparatus for treatment of breast pain using magnetic fields for peripheral nerve stimulation, the apparatus comprising:
a housing unit, the housing unit further comprising:
a power supply;
power supply circuitry;
a controller or microprocessor;
an energy storage capacitor;
a pulse discharge circuitry;
a feedback circuitry; and
a device user interface;
a holster configured to fit around shoulder and chest;
a cable connecting the housing unit to holster;
one or more stimulation coils in the holster configured to stimulate:
the lateral or medial pectoral nerves;
the intercostal brachial nerve (ICBN), the long thoracic nerve (LTN) or the thoracodorsal nerve (TDN); and
the intercostal nerves;
wherein the stimulation coils are placed in a location that is in proximity to the skin to provide optimal stimulation;
wherein the one or more stimulation coils are configured to generate a varying current through the coils which will emit varying magnetic fields to stimulate the peripheral nerves.

20. The apparatus of claim 19 wherein the holster is padded with adhesive.
